(19) United States
(12) Patent Application Publication
(10) Pub. No.: US 2023/0397954 A1
(43) Pub. Date: Dec. 14, 2023

(54) SYSTEMS AND METHODS FOR UTILIZING LASER ABLATION TO MODIFY HEART VALVE PROSTHETIC LEAFLET TISSUE TO IMPROVE BLOOD FLOW

(71) Applicant: University of Denver, Denver, CO (US)

(72) Inventor: Ali Nejatbakhsh Azadani, Denver, CO (US)

(73) Assignee: University of Denver, Denver, CO (US)

(21) Appl. No.: 18/209,170

(22) Filed: Jun. 13, 2023

Related U.S. Application Data
(60) Provisional application No. 63/351,687, filed on Jun. 13, 2022.

(51) Int. Cl. A61B 18/24; A61B 34/00; A61F 2/24

(52) U.S. Cl. CPC A61B 18/24; A61B 34/25; A61F 2/2427; A61B 2018/00369

(57) ABSTRACT

A method for improving blood flow in a patient who has undergone a first heart valve replacement procedure followed by a second heart valve replacement procedure is disclosed. The method includes positioning a second prosthetic replacement valve to replace a primary prosthetic replacement valve within the patient, providing a laser fenestration system including a laser fiber, guiding the laser fiber through the patient's circulatory system to the second prosthetic replacement valve, performing a laser ablation process using the laser fiber to remove portions of a leaflet tissue between any stent structure of the primary prosthetic replacement valve, removing the laser fiber from the patient's circulatory system, and finalizing the second prosthetic valve replacement procedure. In embodiments, the method includes removing native leaflet tissue using laser ablation. In embodiments, a fenestration system for implementing the described method is disclosed.

### REFERENCE TO RELATED APPLICATIONS

The present application claims the benefit of U.S. Provisional patent application Ser. No. 63/351,687, filed 2022 Jun. 13 and titled “Systems and Methods for Utilizing Laser Ablation to Modify Heart Valve Prosthetic Leaflet Tissue to Improve Blood Flow.” The above referenced application is incorporated hereby in its entirety by reference.

### FIELD OF THE INVENTION

The present invention relates generally to heart valve prosthetics. In particular, not by way of limitation, the present disclosure relates to systems and methods to modify heart valve prosthetic leaflet tissue to improve blood flow.

### DESCRIPTION OF RELATED ART

Heart valve replacement procedures are an established treatment for patients with heart diseases affecting the valves including, for example, Aortic Stenosis (AS). It is known and recognized that, with AS, calcium build-up over time narrows the aortic valve opening of the heart, thereby restricting blood flow therethrough. While AS patients generally experience mild symptoms in the early stages, more progressed AS can cause congestive heart failure, sudden loss of consciousness, and even sudden death.

As such, several procedures have been developed for the treatment of AS, with the most prevalent and successful approach being the Transcatheter Aortic Valve Replacement (TAVR). Commonly referred to as the gold standard for treating AS, TAVR involves replacing a diseased aortic valve with a man-made artificial heart valve or heart valve prosthetic.

To date, there are two common designs for these heart valve prosthetics, namely the intra-annular design and the supra-annular design. Placement of either design of prosthetic may be achieved by guiding a catheter through the femoral artery of a patient and up to the patient's aortic valve. Alternatively, the prosthetic may be implanted by creating a small incision in the chest wall of the patient and guiding the prosthetic through the apex of the heart and into the patient's aortic valve. While initially effective, it is known that the heart valve prosthetics used in TAVR procedures may fail over time and require replacement. One of the most common solutions is to perform a valve-in-valve implantation procedure, also referred to as a redo-TAVR, which simply places a second prosthetic within the primary, failing heart valve prosthetic. For some patients, however, the second heart valve prosthetic may cause unanticipated sinus sequestration, which can result in a coronary obstruction.

Sinus sequestration in patients who have undergone a valve-in-valve implantation procedure typically occurs due to the pinning of leaflet tissue from the primary heart valve prosthetic against the stent structure of the prosthetic when the second heart valve prosthetic is implanted within the first. To resolve this issue, a procedure called bioprosthetic or native aortic scallop intentional laceration to prevent iatrogenic coronary artery obstruction (BASILICA) has been developed. During the BASILICA procedure, intervention cardiologists use one or more electrified wires to incise, or slice, the old leaflets of the primary heart valve prosthetic to improve blood flow to the coronary arteries.

However, while the BASILICA procedure may be an option for many valve-in-valve implantation procedure patients, there are known complications pertaining to the commissural alignment between the native and prosthetic valves. This alignment is critical in determining the efficacy of a BASILICA procedure because a misalignment renders the implantation procedure ineffective. For example, in cases where there is an overlap between the first valve prosthetic commissural posts and the coronary ostia, the BASILICA procedure is unlikely to improve blood flow into the coronary sinus.

Thus, there is a need for improved systems and methods of treatment for valve-in-valve prosthetic implantation recipients suffering from pinned leaflet tissue resulting in decreased blood flow, sinus sequestration, or coronary obstruction.

### SUMMARY OF THE INVENTION

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

Some aspects disclosed herein address the above stated needs with systems, methods, and apparatuses for utilizing laser ablation to enhance blood flow through transcatheter valve prosthetics. In an embodiment, a method includes identifying a patient who has undergone a second prosthetic valve replacement procedure is presenting with, or is at risk of presenting with, coronary obstruction or decreased blood flow as a result of the placement of the second valve prosthetic. The method further comprises accessing the circulatory system of the patient, guiding a laser fiber through the patient's circulatory system to the primary and second prosthetic valves, applying laser ablation to remove portions of the primary prosthetic valve leaflet tissues between the stent structure of the primary prosthetic valve, and removing the laser fiber from the patient's circulatory system.

Aspects also include identifying a patient who has undergone a second prosthetic valve replacement procedure and is presenting with, or is at risk of presenting with, coronary artery obstruction or decreased blood flow as a result of the placement of the second valve prosthetic. The method further includes accessing the circulatory system of the patient, guiding a laser fiber through the patient's circulatory system to the primary and second prosthetic valves, identifying regions of overlap between the commissural posts of the primary prosthetic valve and an ostium, applying laser ablation to remove portions of the primary prosthetic valve leaflet tissues between the stent structure of the primary prosthetic valve, and removing the laser fiber from the patient's circulatory system.

According to another aspect, a method includes identifying a patient who has undergone a redo-TAVR procedure. The method further includes accessing the circulatory system of the patient, guiding a laser fiber through the patient's circulatory to the transcatheter aortic valve (TAV) replacement within an existing TAV (i.e., TAV-in-TAV) or TAV replacement within an existing surgical aortic valve (SAV) (i.e., TAV-in-SAV) prosthetics. The method also includes identifying regions of overlap between the commissural posts of the primary TAV prosthetic and the coronary ostia of the patient, applying laser ablation to remove portions of the TAV-in-TAV or TAV-in-SAV prosthetic leaflet tissues between the stent structures of the prosthetic valves, and removing the laser fiber from the patient's circulatory system.

In other aspects, the methods may be applied to other transcatheter heart valves (THVs), such as mitral, pulmonary, and tricuspid positions.

In an embodiment, a method for improving blood flow in a patient who has undergone a first heart valve replacement procedure followed by a second heart valve replacement procedure is disclosed. The method may include positioning a second prosthetic replacement valve to replace a primary prosthetic replacement valve within the patient, providing a laser fenestration system including a laser fiber, guiding the laser fiber through the patient's circulatory system to the second prosthetic replacement valve, performing a laser ablation process using the laser fiber to remove portions of a leaflet tissue between any stent structure of the primary prosthetic replacement valve, removing the laser fiber from the patient's circulatory system, and finalizing the second prosthetic valve replacement procedure.

In another embodiment, a method for improving blood flow after in a patient who has undergone a heart valve replacement procedure includes positioning a prosthetic replacement valve to replace a native valve within the patient, providing a laser fenestration system including a laser fiber, guiding the laser fiber through the patient's circulatory system to the prosthetic replacement valve, performing a laser ablation process using the laser fiber to remove portions of a leaflet tissue of the native valve, removing the laser fiber from the patient's circulatory system, and finalizing the prosthetic valve replacement procedure.

In still another embodiment, a system for improving blood flow in a patient who has undergone a heart valve replacement procedure is disclosed. The system includes a prosthetic replacement valve configured to replace a primary valve and to be positioned within the patient, and a laser fenestration system including a laser fiber. The laser fiber is adapted to be guided through the patient's circulatory system to the prosthetic replacement valve positioned within the patient. The laser fenestration system is adapted to perform a laser ablation process using the laser fiber to remove portions of a leaflet tissue between any stent structure of the prosthetic replacement valve, and the laser fiber is further adapted to be removable from the patient's circulatory system following the laser ablation procedure.

In an embodiment, a fenestration system for user by a user in performing a laser ablation process on a patient in association with a heart valve replacement procedure is disclosed. The fenestration system may include a catheter, a laser source for producing laser energy, an optical fiber for delivering the laser energy to the patient through the catheter, a controller for controlling the laser source, and a user interface for receiving input from the user in operating the fenestration system. The catheter may be configured for guiding the optical fiber through the patient's circulatory system to a heart valve of the patient.

In embodiments, the fenestration system may further include a monitoring system for monitoring at least one of operating conditions of the laser source, a power output of the laser source, and blood flow conditions associated with the heart valve replacement procedure, wherein the monitoring system includes at least one of a temperature sensor, a current meter, a voltmeter, a power meter, a pressure sensor, a flow sensor, and a spectrometer.

In further embodiments, the fenestration system further includes a flush mechanism for providing a liquid flush through the catheter at the fenestration site, wherein the flush mechanism includes a liquid reservoir, a pump, and tubing. In certain embodiments, the flush mechanism is a saline flush mechanism.

In other embodiments, the fenestration system further includes a steering cable connected with the user interface to enable the user to actively control a distal curvature of the catheter.

In yet another embodiment, a method for manufacturing a fenestration system for performing a laser ablation process on a patient in association with a heart valve replacement procedure is disclosed. The method includes providing a laser source for producing laser energy, the laser source being configured for delivering the laser energy through the optical fiber, providing a catheter configured for supporting the optical fiber therein, providing a controller for controlling the laser source, and providing a user interface for receiving input from a user and transmitting the input to the controller in operating the fenestration system. The controller includes a memory for storing machine readable instructions and a processor for executing the machine-readable instructions. In embodiments, the controller is further configured for enabling delivery of the laser energy at a user-specified location within the patient in association with the heart valve replacement procedure.

In certain aspects, the method further includes providing a monitoring system for monitoring at least one of operating conditions of the laser source, a power output of the laser source, and blood flow conditions associated with the heart valve replacement procedure. The monitoring system may include at least one of a temperature sensor, a current meter, a voltmeter, a power meter, a pressure sensor, a flow sensor, and a spectrometer.

In other aspects, the method further includes providing a flush mechanism for providing a liquid flush through the catheter, wherein the flush mechanism includes a liquid reservoir, a pump, and tubing.

In a further aspect, the method further includes providing a steering cable connected with the user interface and the optical fiber, and further configuring the catheter for supporting the steering cable therein. The steering cable and the user interface may be configured to cooperate to enable the user to actively control a distal curvature of the optical fiber in delivering the laser energy at the user-specified location.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF DRAWINGS

FIG. 1 is a flowchart depicting a method of an embodiment of the present disclosure.

FIG. 2 is a flowchart depicting an alternative method of an embodiment of the present disclosure.

FIG. 3 is a flowchart depicting a method of an embodiment of the present disclosure for a patient who has undergone a redo-TAVR procedure.

FIG. 4 is a front view of an exemplary supra-annular transcatheter aortic valve prosthetic.

FIG. 5 shows exemplary valve-in-valve prosthetics with a lacerated valve leaflet from an executed BASILICA procedure and portrays increased blood flow through the lacerated valve leaflet and into the coronary ostia.

FIG. 6A shows a top-view of cross-sectioned aorta portraying commissural alignment between native and bioprosthetic aortic valves.

FIG. 6B shows overlap between the primary prosthetic valve commissural posts and the coronary ostia.

FIG. 7 shows the stent structure of an exemplary valve-in-valve prosthetic with laser ablated leaflet tissue in the stent structure.

FIG. 8 shows an exemplary fiber laser system suitable for use in implementing the methods as described herein, in accordance with embodiments.

FIG. 9 shows a flowchart depicting another exemplary method, in accordance with embodiments.

FIG. 10 shows a simplified diagram of an aortic valve with native calcified leaflets, a transaortic valve frame (i.e., stent), and transaortic valve leaflets.

FIG. 11 shows a simplified diagram of an example of a commercial transcatheter aortic valve.

FIG. 12 shows a simplified diagram of a second example of a commercial transcatheter aortic valve.

FIG. 13 shows a simplified diagram of the leaflets of a transcatheter aortic valve as positioned within the patient.

FIG. 14 shows a simplified diagram of the leaflets of FIG. 13 after laser ablation, in accordance with an embodiment.

FIG. 15 shows a simplified diagram of the leaflets of another transcatheter aortic valve as positioned within the patient.

FIG. 16 shows a simplified diagram of the leaflets of FIG. 15 after laser ablation, in accordance with an embodiment.

FIG. 17 shows a flowchart depicting another exemplary method, in accordance with embodiments.

FIG. 18 shows a simplified diagram of a catheter suitable for use with certain embodiments of the present disclosure.

FIG. 19 shows an exemplary catheter system suitable for use with certain embodiments of the present disclosure.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The flowcharts and block diagrams in the figures illustrate the operation of possible implementations of systems and methods according to various embodiments of the present disclosure. In this regard, in some alternative implementations, the steps noted in the block may occur out of the order noted in the figures. For example, two blocks shown in succession may, in fact, be executed substantially concurrently, or the blocks may sometimes be executed in the reverse order, depending upon the functionality involved. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

### DETAILED DESCRIPTION OF THE INVENTION

The present disclosure relates generally to heart valve prosthetics. In particular, but not by way of limitation, the present disclosure relates to systems and methods for utilizing laser ablation to modify heart valve prosthetic leaflet tissue to improve blood flow through the heart valve prosthetic.

While the BASILICA approach described above has been shown to be effective, a new leaflet modification approach based on laser ablation would be desirable to prevent coronary obstruction and maintain coronary access in all at-risk patients. The new approach may include guiding a laser fiber catheter to the primary (initial) TAV and applying the laser to remove portions of leaflet tissue of the failed TAV or even native valve leaflets as needed. Such a process may be referred to as fenestration.

Beyond improving blood flow, the embodiments described herein enable the addressing of conditions such as:
(1) Commissure Misalignment: Commissural alignment of the initial (index) TAV is essential for success with the existing leaflet modification methods such as BASILICA and others. However, commissural misalignment is common in patients undergoing TAVR. The laser ablation approach can remove portions of leaflet tissue in front of the coronary artery ostium, even in the presence of commissural misalignment, thus aiding in the prevention of coronary obstruction and maintain coronary access in all at-risk patients.
(2) Eccentric Coronary Ostia: In BASILICA and similar procedures, the lacerated leaflet gap enables blood to flow into the coronary artery via flow through the space between the transcatheter heart valve and the sinus of valsalva. Therefore, the exact location of the coronary artery ostium in relation to the cusp is crucial in procedural success. However, if the leaflet gap is not positioned in front of the coronary ostium because of eccentric coronary ostia, flow obstruction may occur, especially when the leaflet is deflected very close to the aortic wall. The laser ablation approach, however, does not have the limitation, and multiple openings may be created in front of the coronary ostia.
(3) Presence of Leaflet Calcification: The target leaflet must be calcium free for success with the existing leaflet modification methods such as BASILICA. It is recognized that confluent heavy leaflet calcification at the nadir of the target aortic leaflets may be an obstacle to leaflet traversal using radiofrequency energy as provided by existing methods. In addition, bulky calcific masses on distal leaflet surfaces can cause coronary obstruction. The laser ablation approach described herein does not have limitation, such as demonstrated by laser atherectomy has been successfully used in the past to treat calcified coronary lesions.
(4) Ability to Create Openings in Tissue and Fabric Skirt: In a low coronary ostium, the fabric-covered frame or skirt can directly obstruct the coronary artery ostium. BASILICA and similar procedures are not suitable for coronary obstruction caused by TAV fabric skirt. None of the existing leaflet modification methods can create openings in the fabric skirt. However, the laser ablation approach described can create openings in leaflets (i.e., pericardium tissue), fabric skirts, and a combination of both as seen in second valve replacement procedures such as redo-TAVR.
(5) Ability to Reduce Blood Stasis in the Neo-sinus and Sinuses: Computational and bench-top studies have pointed to the potential role of blood stasis in neo-sinus in developing hypoattenuated leaflet thickening (HALT) and subclinical leaflet thrombosis. Creating openings in the leaflets of the initial (index) TAV by laser ablation can, for example, (i) increase blood velocity in the neo-sinus and sinus, (ii) reduce blood flow stasis, and (iii) potentially decrease the risk of valve thrombosis.
(6) Reduction in Procedural Time and Contrast Volume: BASILICA and similar approaches are complex procedures. The average time for solo BASILICA (catheter introduction to laceration) is 73 minutes (generally ranging from 58 to 88 min) and for doppio BASILICA is 123 min (generally ranging from 106 to 137 min). The mean total contrast volume in BASILICA is generally around 143 ml (ranging from 101 to 226 ml). The laser ablation approach has the potential to reduce the procedural time and contrast volume.

From a biomechanical point of view, residual stress in native leaflets contributes significantly to the leaflet splay in BASILICA (i.e., the native valve leaflets spring open immediately due of residual stress in living tissue). However, there is no residual stress in pericardium sheets used to fabricate TAV leaflets. Consequently, BASILICA and similar methods are less effective in bioprosthetic heart valves (both surgical and TAVs) than native valves. Besides, newer-generation TAVs are designed with a redundant leaflet for better coaptation, which further limits the splay of these leaflets after the BASILICA and similar methods. Therefore, the laser ablation technique described herein may be significantly superior to the existing methods in preventing coronary obstruction and maintaining coronary access. Considering the history of laser atherectomy in percutaneous coronary intervention, the proposed leaflet modification technique using laser ablation should be safe. In addition, the reported risk for coronary obstruction is probably underestimated because of underdiagnosis when the presentation is atypical and because some high-risk patients were excluded from TAVR because of this potential complication. The new procedure can be expanded to other transcatheter heart valve interventions such as in (i) native aortic valves, (ii) failed surgical aortic bioprostheses, and (iii) other interventions in mitral, tricuspid, and pulmonic positions. This project is anticipated to be a prelude to future animal and clinical studies.

In real-world clinical practice, establishing perpendicular laser contact with the TAVs may not always be feasible. Therefore, the ablation laser may be held at angles, such as 30° to 90° with respect to the tissue to be ablated. For example, a neodymium-yttrium-argon-garnet (Nd:YAG) laser (operating at a wavelength of 1060 nm) may be used to create the fenestration because (i) it is more effective than an excimer laser in laser fenestration, (ii) thermal damage to the surrounding tissue (i.e., the index valve) is not a concern, and (iii) calcium remains an area of challenge for excimer lasers. Other laser systems may be used to provide the laser ablation, and the continuously growing selection and increasing power output of diode lasers may offer a relatively low-cost opportunity to develop laser-assisted therapeutic devices.

It is recognized herein that computational simulations may be used for modeling of blood flow dynamics with quantitative analysis of flow parameters, such as velocity and blood stasis, in the proximity of the TAV leaflets. Recognizing the variety of parameters influencing blood flow, a fluid-structure interaction (FSI) modeling approach may be used to simulate unsteady flow fields of bioprosthetic heart valves to be integrated into the implementation of the laser ablation-based methods described herein. Additionally, the three-dimensional unsteady flow fields of the valves may also be compared with in vitro tests using FSI simulations to further investigate flow patterns and determine the contours of blood residence time, quantity shear stress, shear stress gradient, and oscillatory shear index in neo-sinus and on the surface of the leaflets, thus further refining the laser operations in accordance with certain embodiments.

The laser ablation processes described herein may result in an increased velocity in the sinus and neo-sinus due to the ablated openings improving blood flow in the sinus and neo-sinus, thus potentially reducing the risk of subclinical leaflet thrombosis.

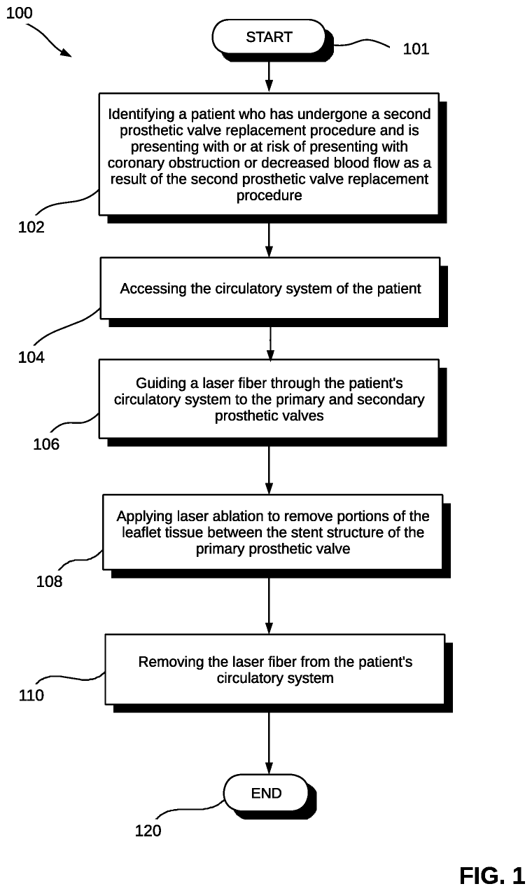

FIG. 1 is a flowchart depicting a method 100 of an embodiment of the present disclosure Method 100 begins with a start step 101, and includes identifying a patient who has undergone a second prosthetic valve replacement procedure and is presenting with or at risk of presenting with coronary obstruction or decreased blood flow as a result of the placement of the second heart valve prosthetic (block 102); accessing the circulatory system of the patient (block 104); guiding a laser fiber through the patients circulatory system to the primary and second prosthetic valves (block 106); applying laser ablation to remove portions of the primary and second prosthetic valve leaflet tissues between the stent structure of the prosthetic valves (block 108); and removing the laser fiber from the patient's circulatory system (block 110). Method 100 terminates in an end step 120.

It is noted that the use of laser ablation to remove the unwanted tissue provides a variety of advantages over existing methods (e.g., leaflet tear using electrified wires) such as, not limited to, more precise control over the specific tissue removed in the procedure, accurate delivery of ablation energy, reduced likelihood of damage to the tissues of the heart and surroundings, and reduced patient trauma and recovery time due to the small incision required for arterial delivery of the laser fiber to the prosthetic area. While endovenous laser ablation has been used in the treatment of conditions such as varicose veins, the use of laser energy delivery via, for example, a laser catheter for heart surgery has thus far been limited to more diffuse delivery of laser energy such as for treatment of persistent atrial fibrillation (see, for example, Weber, et al. “Laser catheter ablation of long-lasting persistent atrial fibrillation: Longterm results,” J. Atr. Fibrillation, Vo. 10, No. 2, 2017, https://www.ncbi.nlm.nih.gov/pmc/articles/PMC5673290/accessed 2023-06-08). In the present disclosure, the laser fiber enables pinpoint accuracy in the delivery of laser energy for the precise removal of specific portions of the aortic leaflets after the replacement valve prosthetic has been positioned in the desired location to improve blood flow into the coronary artery. That is, in the embodiments disclosed herein, the goal is to create one or more openings at strategic locations in the tissue related to heart valves, thus is distinct the atrial fibrillation ablation of Weber, which instead creates scars in the heart to block the faulty electrical signals. Further, laser ablation may be used with both stented and stentless prosthetic valves without damaging the stent, if used.

In some embodiments the identified patient may have undergone a surgical aortic valve replacement (SAVR) procedure and may have received any heart valve prosthetic, such as a stented prosthetic valve, a stented, supra-annular position prosthetic valve, a stented, externally mounted leaflet prosthetic valve, or a stentless prosthetic valve, to name a few nonlimiting examples.

In some embodiments, the identified patient may have undergone a mitral valve replacement (MRV) procedure and may have any received any heart valve prosthetic, such as a stented prosthetic valve, a stented, supra-annular position prosthetic valve, a stented, externally mounted leaflet prosthetic valve, or a stentless prosthetic valve, to name a few nonlimiting examples.

In some embodiments, the identified patient may have undergone a transcatheter pulmonary valve replacement (TPRV) procedure and may have any received any valve prosthetic.

In some embodiments, the identified patient may have undergone a tricuspid valve replacement procedure and may have received any valve prosthetic.

In some embodiments the laser fiber is guided through a patient's circulatory system by means of a guide wire and/or a catheter.

In some embodiments the method may be executed by means of open-heart surgery or through other means of accessing a patient's heart valves.

In some embodiments the patient may be asymptomatic, or not present with or be at risk of presenting with coronary obstruction or decreased blood flow as a result of the placement of a second heart valve prosthetic.

In some embodiments laser ablation may be utilized to remove portions of heart valve leaflet tissue from either the primary or second heart valve prosthetic.

In some embodiments the method may be applied to other transcatheter heart valves (THVs), such as a mitral, pulmonary, and tricuspid positions.

In some embodiments the method can also be used in transcatheter heart valves to improve blood flow in the vicinity of the leaflets to reduce blood stasis and thereby minimize the risk of leaflet thrombosis.

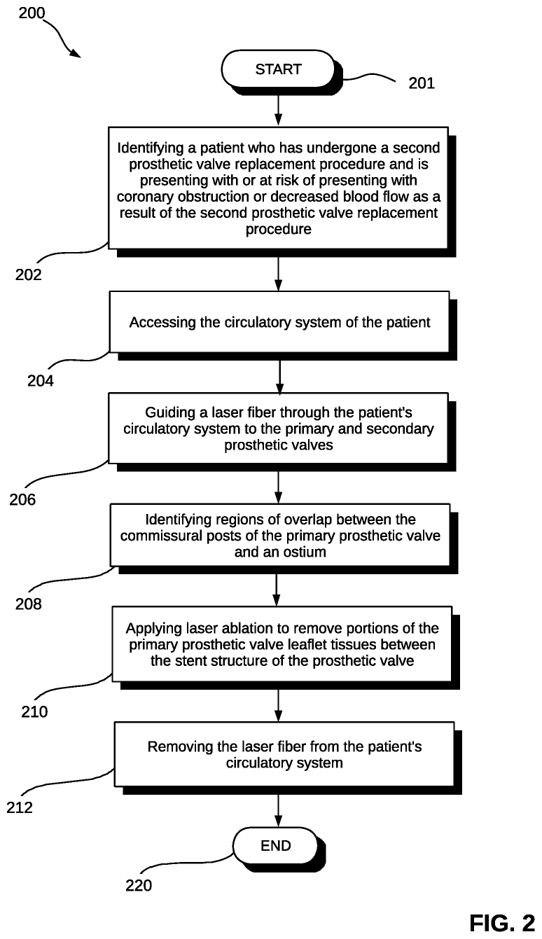

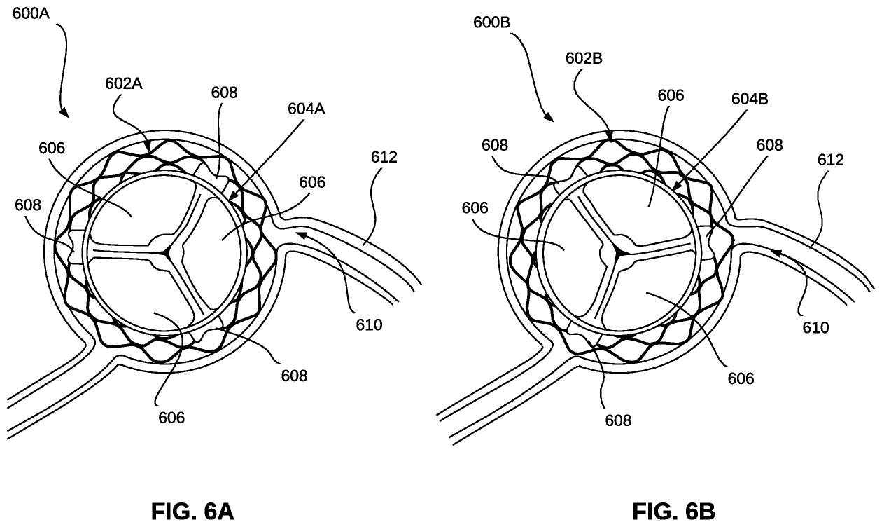

FIG. 2 is a flowchart depicting an alternative method 200 of an embodiment of the present disclosure. Alternative method 200 begins with a start step 201, then proceeds to identifying a patient who has undergone a second prosthetic valve replacement procedure and is presenting with or at risk of presenting with coronary obstruction or decreased blood flow as a result of the placement of the second valve prosthetic (block 202); accessing the circulatory system of the patient (block 204); guiding a laser fiber through the patients circulatory system to the primary and second prosthetic valves (block 206); identifying regions of overlap between the commissural posts of the primary prosthetic valve and an ostium (block 208); applying laser ablation to remove portions of the primary and second prosthetic valve leaflet tissues between the stent structure of the prosthetic valves (block 210); and removing the laser fiber from the patient's circulatory system (block 212). Alternative method 200 terminates in an end step 220. The step of identifying regions of overlap between the commissural posts and an ostium is described in further detail below with respect to FIGS. 6A and 6B. It is noted that ablation via laser fiber as described herein enables a more precise delivery of the ablation energy compared to, for instance, the BASILICA procedure, thus enabling safer removal of the prosthetic valve leaflet tissues with a reduced likelihood of damage to the aorta and the surrounding heart tissue.

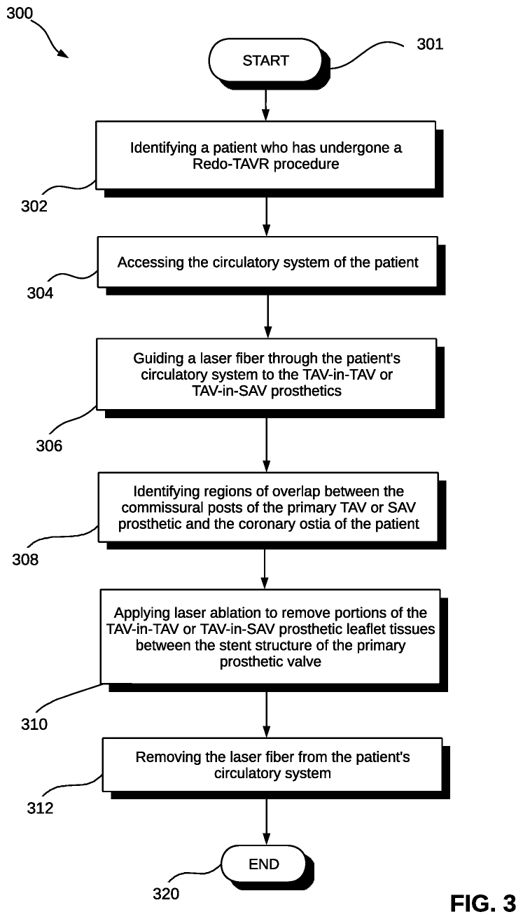

FIG. 3 is a flowchart depicting a method 300 of an embodiment of the present disclosure for patients who have undergone a redo-TAVR procedure. Method 300 begins with a start step 301, then proceeds to identifying a patient that underwent a Redo-TAVR procedure (block 302); accessing the circulatory system of the patient (block 304); guiding a laser fiber through the patients circulatory system to the TAV-in-TAV or TAV-in-SAV prosthetics (block 306); identifying regions of overlap between the commissural posts of the primary TAV prosthetic and the coronary ostia of the patient (block 308); applying laser ablation to remove portions of the TAV-in-TAV or TAV-in-SAV prosthetic leaflet tissues between the stent structures of the prosthetic valves (block 310); and removing the laser fiber from the patient's circulatory system (block 312). Method 300 terminates in an end step 320.

It is noted that, while each one of the embodiments illustrated in FIGS. 1-3 is shown to begin with a step to identify a particular type of patient who has undergone a second prosthetic valve replacement procedure, this identification step may be integrated into the second prosthetic valve procedure itself. That is, the steps that follow the identification step in each of methods 100, 200, and 300 may be performed immediately following the placement of the aortic valve prosthetic, in accordance with certain embodiments. In other words, a laser fiber ablation system may be used by the operating cardiologist/surgeon as a part of the second prosthetic valve replacement procedure, thus improving patient outcomes rather than not performing the laser ablation.

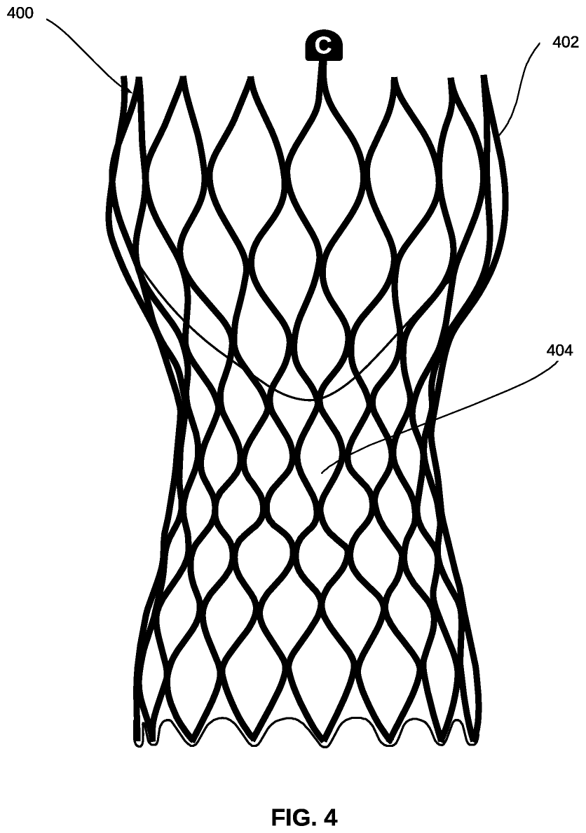

FIG. 4 illustrates a front view of an exemplary supra-annular transcatheter aortic valve (TAV) prosthetic 400 with a stent structure 402 and leaflet tissues 404. While implementation of TAV prosthetic 400 may be an indicator of a patient at risk of coronary obstruction, patients with other types of valve prosthetics may also benefit from laser ablation as described above to improve blood flow. The laser ablation process described herein is advantageous over existing methods, such as the BASILICA procedure, in that the laser energy delivery via a laser fiber enables a more precise delivery for the ablation, thus reducing the risk of damage to the surrounding heart tissue while improving patient outcomes.

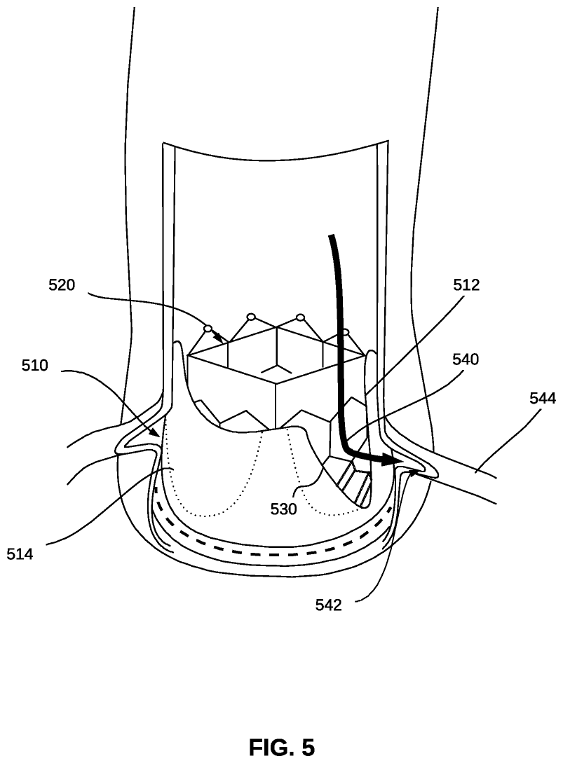

FIG. 5 shows exemplary valve-in-valve prosthetics 500 with a lacerated valve leaflet from an executed BASILICA procedure and portrays increased blood flow through a lacerated valve leaflet and into a coronary ostia (see, for example, J. Khan, et al., “Transcatheter Laceration of Aortic Leaflets to Prevent Coronary Obstruction during Transcatheter Aortic Valve Replacement: Concept to First-in-Human,” JACC: Cardiovascular Interventions, vol. 11, No. 7, 2018 (https://www.jacc.org/doi/10.1016/j.jcin.2021.02.035 accessed 2023-06-07) and J. Khan, et al., “Preventing Coronary Obstruction during Transcatheter Aortic Valve Replacement: Results from the Multicenter International BASILICA Registry,” JACC: Cardiovascular Interventions, vol. 14, no. 9, 2021 (https://www.researchgate.net/publication/324175734_Transcatheter_Laceration_of_Aortic_Le aflets_to_Prevent_Coronary_Obstruction_During_Transcatheter_Aortic_Valve_Replacement, accessed 2023-06-07)). As shown in FIG. 5, valve-in-valve prosthetics 500 includes a primary valve prosthetic 510 including a stent 512 and valve leaflets 514. A second valve prosthetic 520 is housed within primary valve prosthetic 510 to form a valve-in-valve configuration. As shown, a laceration 530 is formed in a portion of valve leaflets 514 to enable blood flow (represented by curved arrow 540) into coronary ostia 542 of coronary artery 544.

FIG. 6A shows a top, cross-sectional view of an aorta 600A portraying commissural alignment between native and bioprosthetic aortic valves (FIGS. 6A and 6B have been adapted from Søndergaard, et al., “Transcatheter aortic valve implantation: don't forget the coronary arteries!” Eurointervention: Journal of Europer in Collaboration with the Working Group on Interventional Cardiology of the European Society of Cardiology, 2018; 14:147-149). As shown in FIG. 6A, a stent 602A and a valve prosthetic 604A are placed within aorta 600A. Valve prosthetic 604A includes three leaflets 606 to form a tricuspid leaflet structure, with commissural posts 608 at the locations between the leaflets. In FIG. 6A, valve prosthetic 604A is aligned within aorta 600A such that commissural posts 608 are clear of coronary ostia 610 of coronary artery 612 such that blood may flow freely into coronary ostial 610.

In contrast, FIG. 6B shows a top, cross-sectional view of an aorta 600B portraying overlap between the primary prosthetic valve's commissural posts and the coronary ostia of a patient. Also shown is the tricuspid leaflet structure of the bioprosthetic aortic valve and accompanying stent structure 602B. As shown in FIG. 6B, stent 602B and valve prosthetic 604B are aligned within aorta 600B such that one of commissural posts 608 aligns with coronary ostia 610, thus potentially restricting blood flow into coronary artery 612. In such a situation, the patient may benefit from ablation of a portion of the valve prosthetic leaflets overlapping the coronary ostia.

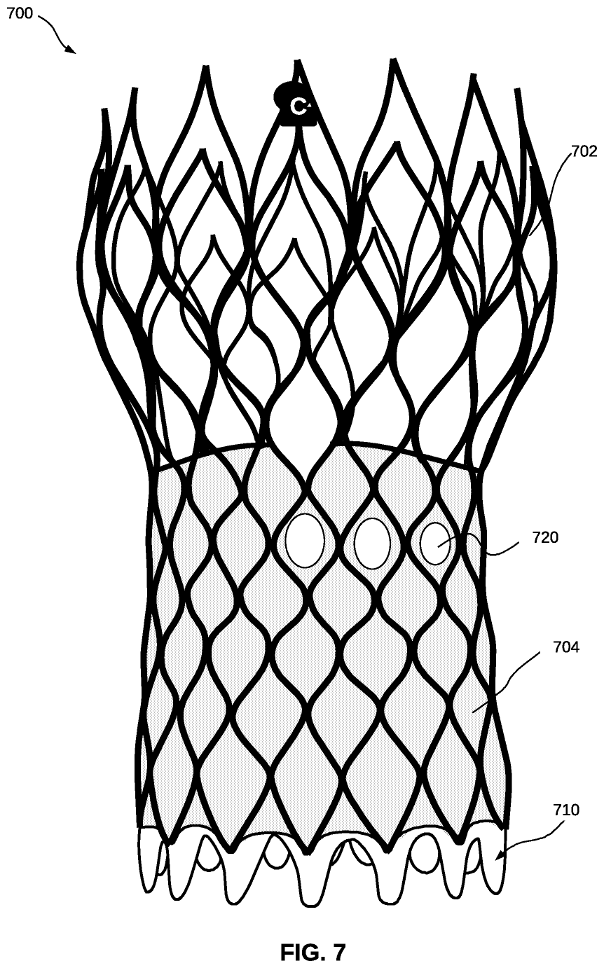

FIG. 7 shows a valve-in-valve prosthetic 700 including a stent structure 702 surrounding leaflet tissue 704, visible between the mesh of stent structure 702, with a second valve prosthetic 710, protruding below stent structure 702. Portions of leaflet tissue 704 may be ablated away by a laser to form openings 720 such that blood flow through prosthetic 700 is not obstructed by leaflet tissue 704 at openings 720.

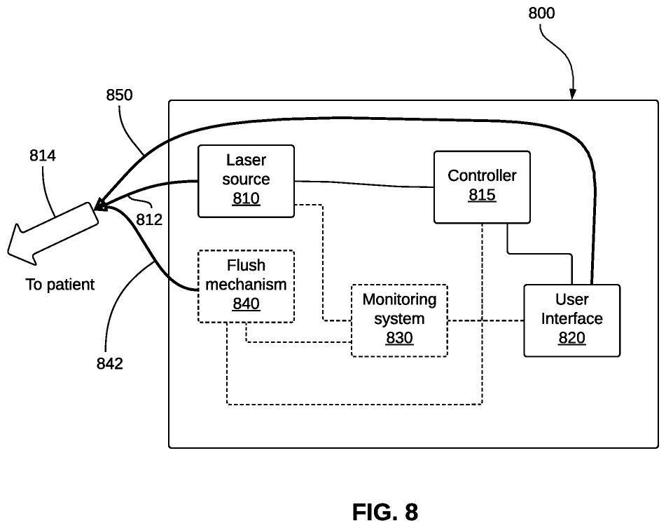

FIG. 8 shows a fenestration tool 800 suitable for use in implementing the methods described herein, in certain embodiments. Fenestration tool may include, for example, a bundle of fiber optic cables and other tubing incorporated into a standard catheter, such as a 10-French steerable catheter). Currently available catheters for laser angioplasty are specially designed for laser angioplasty for coronary arteries, thus are not suitable for use with transaortic valves. For example, in certain embodiments of the fenestration purposes described herein, the tip of the laser fiber and/or catheter should be positionable perpendicular to the surface of the leaflets of the valve. Such positioning may require the catheter to be bendable in ways not achievable with existing catheters. Further, in embodiments, once the proper longitudinal and rotational alignments are reached, the stability of the catheter must be maintained during the fenestration process. Additionally, in embodiments, a flush mechanism, such as a saline flush mechanism including a saline reservoir, pump, and/or tubing to deliver a liquid flush at the fenestration site as needed may be integrated into the fenestration tool. A steering cable may be used to actively control the distal curvature of the catheter. A stabilizer may also be integrated into the catheter for maintaining the position of the catheter as the catheter is advanced through the aortic arch.

As shown in FIG. 8, fenestration tool 800 includes a laser source 810 connected with an optical fiber 812 configured to deliver laser energy from laser source 810 through a catheter (represented by a thick arrow 814) to a selected location (such as at a valve prosthetic leaflet) within a patient. Operations of laser source 810 may be controlled by a controller 815, which may modify various aspects of the laser source, such as power, pulse duration, timing, and other parameters used in controlling the laser energy output from laser source 810. Controller 815 may include, for example, one or more processors for receiving and processing inputs from a user interface 820. User interface 820 may include mechanisms for a user (e.g., a surgeon/cardiologist) to provide input to adjust the laser source operations as well as components such as a display for providing feedback to the user regarding the laser source operations. Optionally, a monitoring system 830 may be incorporated into laser ablation system 800 to monitor and collect, for instance, operational parameters of laser source 810, controller 815, and user interface 820. Monitoring system 830, for example, may be connected with sensors such as temperature sensors, current meters, voltmeters, power meters, pressure sensors, flow sensors, and spectrometers for monitoring the operating conditions and laser power outputs of laser source 810 and/or the blood flow conditions through and around the implanted valve and fenestration sites.

Fenestration tool 800 may further include a flush mechanism 840, which may include a variety of components for implementing a liquid flush via tubing 842 to catheter 814 such as, and not limited to, a liquid reservoir, a connector to an external liquid source, a pump, and delivery tubing. For example, flush mechanism 840 may be a saline flush mechanism for providing targeted saline flush at or near the fenestration site. Additionally, a steering cable 850 may be connected with the user interface and the catheter and/or optical fiber to enable the user of fenestration tool 800 to control a distal curvature of the optical fiber, thus allowing the user to direct the laser energy to a desired location within the patient.

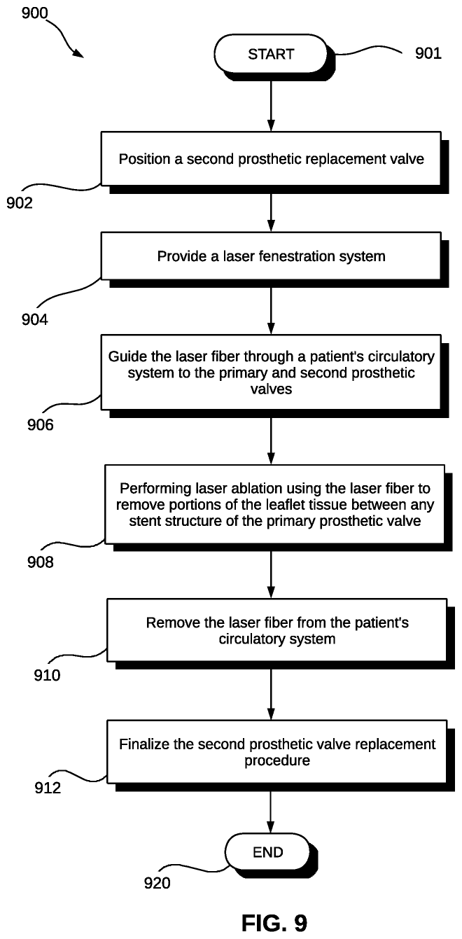

FIG. 9 shows a flow chart illustrating a method 900 of an embodiment of the present disclosure wherein a second prosthetic valve replacement procedure is integrated with the laser ablation procedure described above. Method 900 begins with a start step 901, then proceeds to a step 902 to position a second prosthetic replacement valve. Step 902 may be performed in accordance with procedures such as, for example, TAVR, SAVR, stented or stentless prosthetic valve replacement, and other valve replacement processes known in the art.

Method 900 proceeds to a step 904 to provide a laser fenestration system, such as laser ablation system 800 shown in FIG. 8. Then, method 900 proceeds to a step 906 to guide the laser fiber through a patient's circulatory system to the primary and second prosthetic valves, a step 908 to use the laser fiber to remove portions of the leaflet tissue between any stent structure of the primary prosthetic valve, and a step 910 to remove the laser fiber from the patient's circulatory system. Method 900 concludes with a step 912 to finalize the second prosthetic valve replacement procedure, and terminates in an end step 920.

FIG. 10 shows a simplified diagram 1000 of an aortic valve with native calcified leaflets, a transaortic valve frame (i.e., stent), and transaortic valve leaflets. As shown in FIG. 10, native leaflets 1010 may be calcified such that a transaortic valve is required for maintaining proper heart function. A TAV frame 1020 and TAV leaflets 1030 may be implanted, although the TAV frame and leaflets may be substantially obstructed or covered by native leaflets 1010. In such a case, performing a fenestration process through the native valve leaflets may increase blood velocity in the neo-sinus and sinus, reduce blood flow stasis on the TAV leaflets, and help decrease the risk of valve thrombosis. Due to the calcification, such a fenestration process of the native valve leaflets is not possible using existing processes, such as BASILICA, due to the manner in which the hole-opening energy is delivered using the existing processes. It is recognized herein that the laser ablation process described herein enables such fenestration processes heretofore not possible. Further, the fenestration process described herein may be suitable for use with other transcatheter heart valve interventions in valvular positions such as aortic, mitral, pulmonary, and tricuspid valves including native, bioprosthetic, tissue-engineered, and other types valve mechanisms.

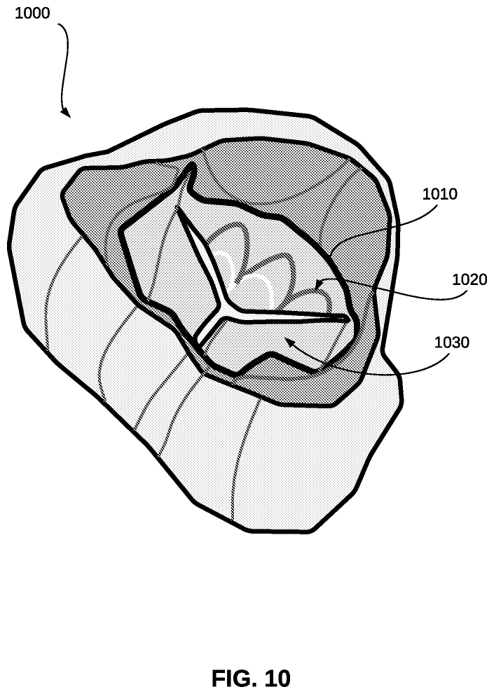

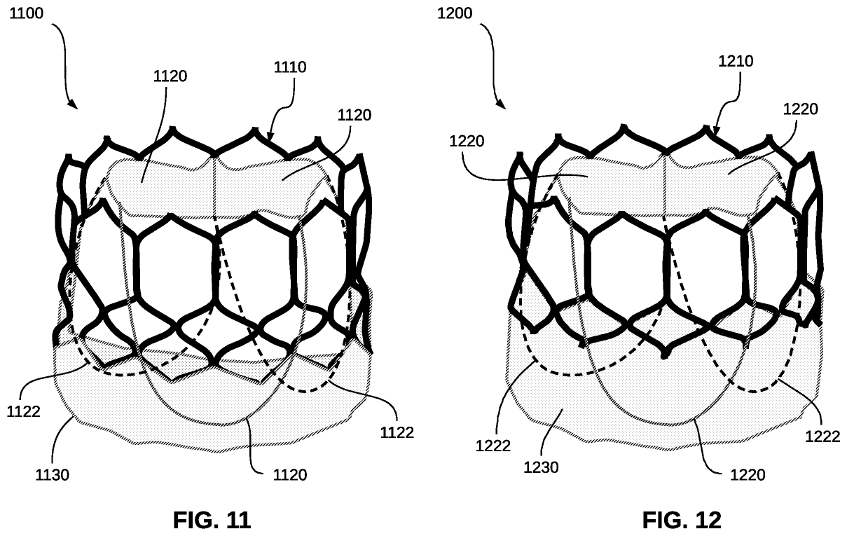

The implementation of the laser ablation fenestration process is described as applied to certain examples of intra- and sura-annular valve devices. For example, FIGS. 11 and 12 show simplified diagrams of the SAPIEN 3 and SAPIEN3 ULTRA valves available from Edwards Lifesciences Corporation.

FIG. 11 shows a simplified diagram of the SAPIEN 3 valve 1100, which is a commercial transcatheter aortic valve. Valve 1100 includes a stent frame 1110 and leaflets 1120 supported therein. The outline of leaflets are represented by dashed curves 1122. Valve 1100 also includes an outer sealing skirt 1130.

FIG. 12 shows a simplified diagram of the SAPIEN 3 ULTRA valve 1200, as a second example of a commercial transcatheter aortic valve. Similar to valve 1100 of FIG. 11, valve 1200 includes a stent frame 1210 and leaflets 1120 (with the shape of the non-visible leaflets represented by dashed lines 1222. Valve 1200 includes an outer sealing skirt 1230, which is taller than outer sealing skirt 1130.

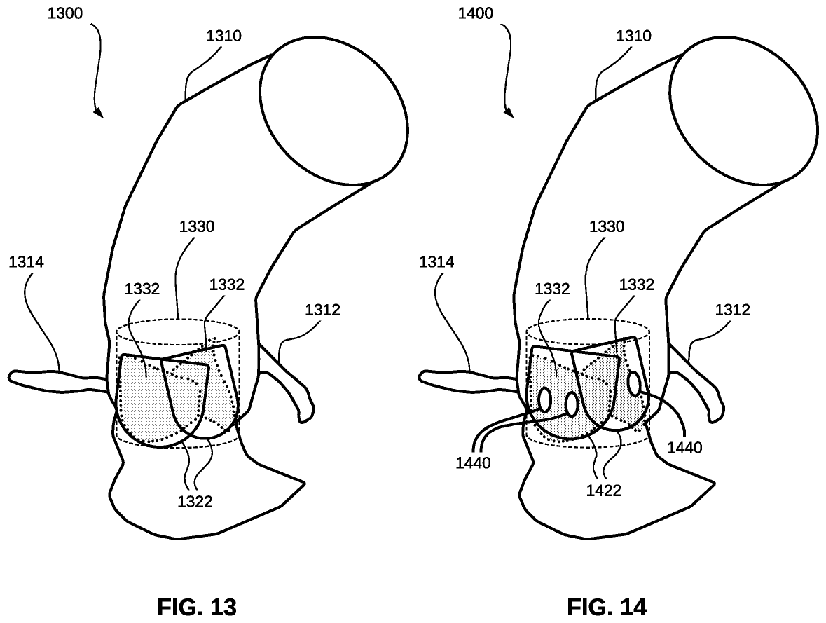

FIG. 13 shows a simplified diagram of the leaflets of a transcatheter aortic valve as positioned within the patient. A diagram 1300 shows a portion of an aorta 1310 with right and left aortic arteries 1312 and 1314, respectively. Native valve leaflets 1322 are shown with a TAV system 1330, such as those shown in FIGS. 11 and 12, implanted therein. As visible in FIG. 13, TAV leaflets 1332 are shown to substantially overlap with native valve leaflets 1322, thus potentially resulting in restricted blood flow. In order to address this issue, a laser ablation fenestration process may be performed to create openings in the native valve leaflets. It is noted that this process may be used with a variety of situations in which an aortic valve replacement has been performed, such as with a TAV in combination of a native valve, a failed surgical aortic valve (SAV) replacement, and a failed TAV.

FIG. 14 shows a simplified diagram of the leaflets of FIG. 13 after laser ablation, in accordance with an embodiment. As shown in a diagram 1400, native valve leaflets 1422 have been modified with holes 1440 formed therethrough in order to improve blood flow. A specialized fenestration system, such as that shown in FIG. 8, may be particularly useful in performing the fenestration procedure.

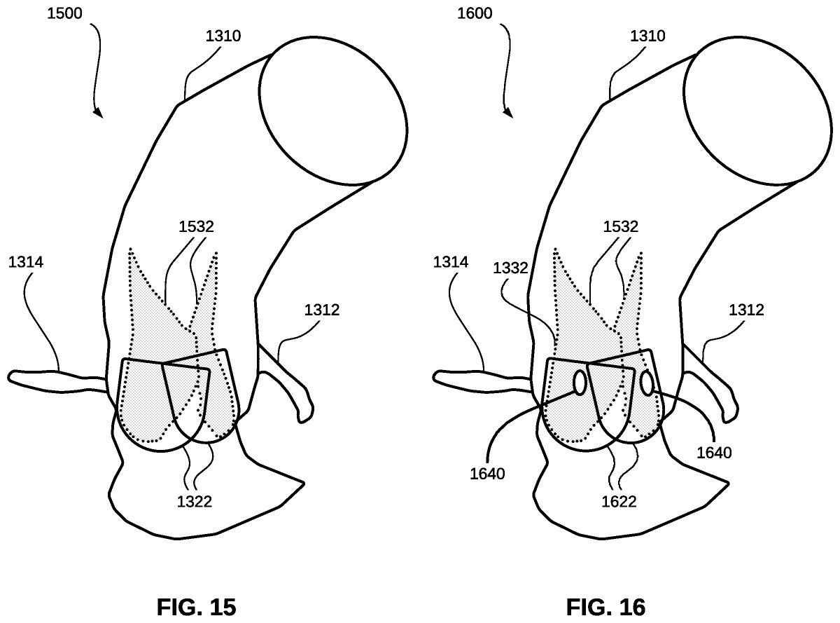

FIG. 15 shows a simplified diagram of the leaflets of another transcatheter aortic valve as positioned within the patient, such as the device shown in FIG. 4. As shown in a diagram 1500, TAV leaflets 1532 are again substantially overlapped with native valve leaflets 1322, such that a fenestration process would be beneficial.

FIG. 16 shows a simplified diagram of the leaflets of FIG. 15 after laser ablation, in accordance with an embodiment. As shown in a diagram 1600, native leaflets 1622 have been modified with holes 1640 formed therethrough by the laser ablation fenestration process described herein.

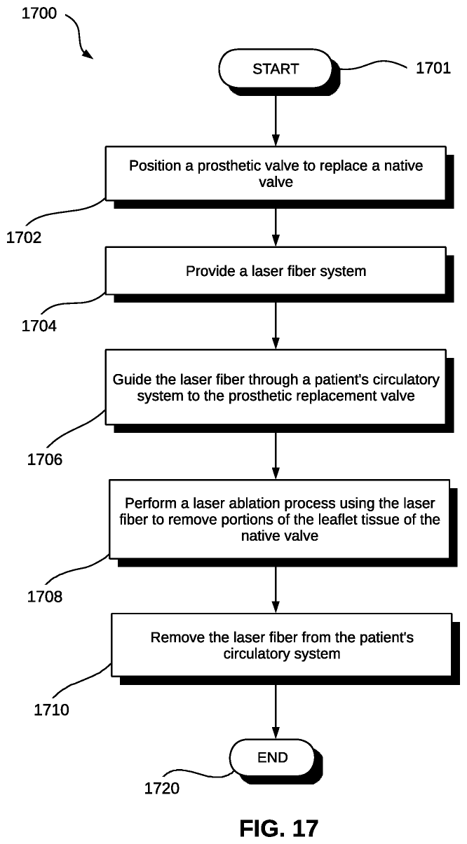

FIG. 17 shows a flow chart illustrating a method 1700 of another embodiment of the present disclosure including laser ablation fenestration process of native valve leaflets. Method 1700 begins with a start step 1701, then proceeds to a step 1702 to position a prosthetic valve to replace a native valve. Step 1702 may be performed in accordance with procedures such as, for example, TAVR, SAVR, stented or stentless prosthetic valve replacement, and other valve replacement processes known in the art. It is noted that step 1702 also may include the positioning of a second prosthetic valve, particularly in cases a native valve or portions thereof (e.g., calcified native leaflets) are still present.

Method 1700 proceeds to a step 1704 to provide a laser fenestration system, such as fenestration system 800 shown in FIG. 8. Then, method 1700 proceeds to a step 1706 to guide the laser fiber through a patient's circulatory system to the prosthetic replacement valve, a step 1708 to use the laser fiber to remove portions of the native leaflet tissue, and a step 1710 to remove the laser fiber from the patient's circulatory system. Method 1700 terminates in an end step 920.

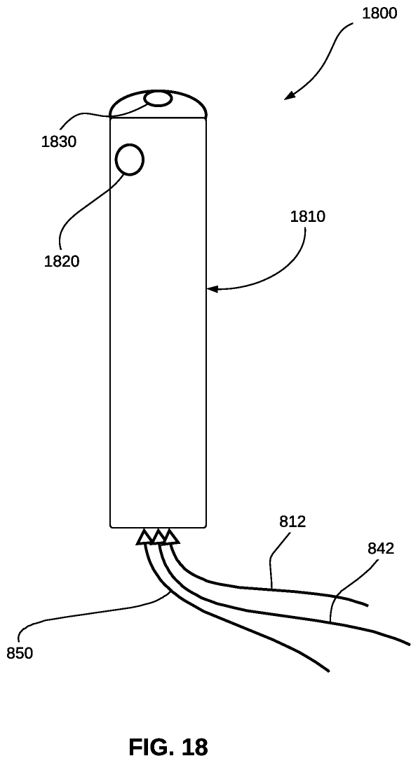

FIG. 18 shows a simplified diagram of a catheter suitable for use with certain embodiments of the present disclosure. As shown, a catheter 1800 includes a catheter body 1810 configured to accommodate components such as optical fiber 812, tubing 842, and steering cable 850 therethrough. For instance, catheter body 1810 may include internal clips for affixing the various components therein. As another example, the various components may be embedded within catheter body 1810 using, for example, epoxy, adhesives, and the like.

In an embodiment, catheter body 1810 includes a window 1820, through which the laser energy from a laser source (e.g., laser source 810 of FIG. 8) may be delivered to the patient. For instance, window 1820 may be formed of a material transparent to the wavelength of the laser energy, while protecting the contents of catheter body 1810. In certain examples, window 1820 may be an end portion of optical fiber 812 itself, such that the end portion of optical fiber 812 may be directly insertable into an opening formed to accommodate window 1820.

An end opening 1830 may also be formed into catheter body 1810 to enable dispersion of flush fluid from tubing 842 therethrough. Alternatively, end opening 1830 may also accommodate the delivery of laser energy from optical fiber 812 therethrough.

While catheter body 1810 is shown in FIG. 18 as a solid piece, it may be formed of a flexible material, such as a flexible tubing as commonly known in the catheter arts (e.g., 10-French steerable catheter). Catheter 1800 is configured for enabling the positioning of the optical fiber, tubing, steering cable, and any other component enclosed therein through the circulatory system of the patient, such as inserted from the femoral artery.

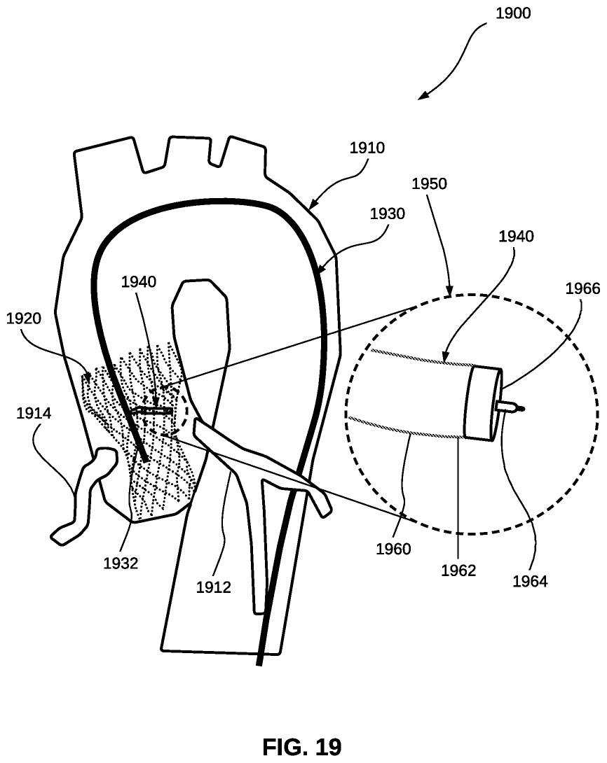

FIG. 19 shows an exemplary catheter system suitable for use with certain embodiments of the present disclosure. A diagram 1900 shows a portion of an aorta 1910 of a patient with a right aortic artery 1912 and a left aortic artery 1914 protruding therefrom. An exemplary transcatheter aortic valve 1920 (e.g., TAV prosthetic 400 of FIG. 4) positioned within aorta 1910. A catheter 1930 may be directed through the patient's circulatory system to reach the location of TAV 1920. In the embodiment illustrated in FIG. 19, catheter 1930 is configured to enable a catheter body 1932 to be separated from a laser delivery portion 1940 when, for example, the user engages a steering cable to adjust a distal curvature of the laser delivery portion (for example, further pulling the optical fiber within laser delivery portion 1940 to be substantially normal to a surface of a leaflet to be ablated). In certain examples, rather than separating catheter body 1932 from laser delivery portion 1940, the entire catheter (including the optical fiber for delivering laser energy therethrough) may be curved to be brought in proximity with the portion of TAV prosthetic (or native valve leaflet) to be ablated.

An inset 1950 shows a close-up view of an example configuration of laser delivery portion 1940. As seen in inset 1950, laser delivery portion 1940 includes a laser tubing 1960 capped with a laser ferrule 1962. Laser ferrule 1962, for example, may be configured for transmitting therethrough the laser energy from one or more optical fibers bundled within laser tubing 1960. Laser ferrule 1962 may be integrated with a flush nozzle 1964 such that laser energy may be transmitted through a surface 1966 around flush nozzle 1964. Other combinations of flush mechanism and laser energy delivery systems may be contemplated and are considered a part of the present disclosure.

It is noted that the fenestration process described above may be performed immediately following a TAVR procedure, if a coronary blockage is observed during the procedure. Alternatively, the fenestration process may be performed separately from the TAVR procedure, if there is a later diagnosed need for coronary access, for example, for coronary stent implantation.

The foregoing is considered as illustrative only on the principles of the disclosure. Further, since numerous modifications and changes will occur to those skilled in the art, it is not desired to limit the disclosure to the exact construction and operation shown and described, and accordingly, all suitable modifications and equivalents may be resorted to, falling within the scope of the disclosure. It is noted that certain aspects of the present disclosure may be performed by robots, such as in robot-implemented surgery methods. Further, certain aspects of the present disclosure may be performed by systems implementing artificial intelligence as trained by machine learning methods.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding”— whether explicitly discussed or not—and, conversely, were there only disclosure of the act of “protruding”, such a disclosure should be understood to encompass disclosure of a “protrusion”. Such changes and alternative terms are to be understood to be explicitly included in the description.

1. A system for improving blood flow in a patient who has undergone a heart valve replacement procedure, the system comprising:
a prosthetic replacement valve configured to replace a primary valve and to be positioned within the patient; and
a laser fenestration system including a laser fiber,
wherein the laser fiber is adapted to be guided through the patient's circulatory system to the prosthetic replacement valve positioned within the patient,
wherein the laser fenestration system is adapted to perform a laser ablation process using the laser fiber to remove portions of a leaflet tissue between any stent structure of the prosthetic replacement valve, and
wherein the laser fiber is further adapted to be removable from the patient's circulatory system following the laser ablation procedure.

2. A fenestration system for use by a user in performing a laser ablation process on a patient in association with a heart valve replacement procedure, the fenestration system comprising:
a catheter;
a laser source for producing laser energy;
an optical fiber for delivering the laser energy to the patient through the catheter;
a controller for controlling the laser source; and
a user interface for receiving input from the user in operating the fenestration system,
the catheter being configured for guiding the optical fiber through the patient's circulatory system to a heart valve of the patient.

3. The fenestration system of claim 2, further comprising:
a monitoring system for monitoring at least one of operating conditions of the laser source, a power output of the laser source, and blood flow conditions associated with the heart valve replacement procedure,
wherein the monitoring system includes at least one of a temperature sensor, a current meter, a voltmeter, a power meter, a pressure sensor, a flow sensor, and a spectrometer.

4. The fenestration system of claim 2, further comprising:
a flush mechanism for providing a liquid flush through the catheter at the fenestration site,
wherein the flush mechanism includes a liquid reservoir, a pump, and tubing.

5. The fenestration system of claim 4, wherein the flush mechanism is a saline flush mechanism.

6. The fenestration system of claim 2, further comprising:
a steering cable connected with the user interface to enable the user to actively control a distal curvature of the optical fiber.

7. A method for manufacturing a fenestration system for performing a laser ablation process on a patient in association with a heart valve replacement procedure, the method comprising:
providing a laser source for producing laser energy, the laser source being configured for delivering the laser energy through the optical fiber;
providing a catheter configured for supporting the optical fiber therein;
providing a controller for controlling the laser source; and
providing a user interface for receiving input from a user and transmitting the input to the controller in operating the fenestration system,
wherein the controller includes a memory for storing machine readable instructions and a processor for executing the machine-readable instructions,
wherein the controller is further configured for enabling delivery of the laser energy at a user-specified location within the patient in association with the heart valve replacement procedure.

8. The method of claim 7, further comprising:
providing a monitoring system for monitoring at least one of operating conditions of the laser source, a power output of the laser source, and blood flow conditions associated with the heart valve replacement procedure,
wherein the monitoring system includes at least one of a temperature sensor, a current meter, a voltmeter, a power meter, a pressure sensor, a flow sensor, and a spectrometer.

9. The method of claim 7, further comprising:
providing a flush mechanism for providing a liquid flush through the catheter,
wherein the flush mechanism includes a liquid reservoir, a pump, and tubing.

10. The method of claim 7, further comprising:
providing a steering cable connected with the user interface and the optical fiber; and
further configuring the catheter for supporting the steering cable therein,
wherein the steering cable and the user interface are configured to cooperate to enable the user to actively control a distal curvature of the optical fiber in delivering the laser energy at the user-specified location.
